(19) United States
(12) Patent Application Publication
(10) Pub. No.: US 2022/0039958 A1
(43) Pub. Date: Feb. 10, 2022

(54) TEMPLATE

(71) Applicant: Shigeyuki OZAKI, Tokyo (JP)

(72) Inventor: Shigeyuki OZAKI, Tokyo (JP)

(21) Appl. No.: 17/281,365

(22) PCT Filed: Apr. 16, 2019
(86) PCT No.: PCT/JP2019/039524

§ 371 Date: May 10, 2021

(30) Foreign Application Priority Data
Oct. 9, 2018 (JP) 2018-190673

(51) Int. Cl. A61F 2/24

(52) U.S. Cl. CPC A61F 2/2496

(57) ABSTRACT

Provided is a template 1 including a plurality of forming parts 2 to accommodate various sizes of valve cusps to be formed from a pericardium. Each of the forming parts 2 includes a hole 10A and a hole 10B for drawing lines which indicate positions at which the pericardium taker from a patient is to be cut. Each of the forming parts 2 further includes a hole 11A, a plurality of holes 11B, holes 11C, and holes 11D for drawing marks that indicate positions at which a valve cusp material cut and formed from the pericardium is sutured to a blood vessel. The lines that indicate the cutting positions are drawn on the pericardium by moving a skin marker 5 along the holes 10A and 10B. The drawing of the marks corresponding to the suture positions at the hole 11A, the holes 11B, the holes 11C, and the holes 11D is carried out with the skin marker 5.

### TECHNICAL FIELD

The present invention relates to a template.

### BACKGROUND ART

As a template for forming an aortic valve from a pericardium, there is, for example, a template disclosed in Patent Document 1. This template includes a valve cusp base forming part, which is a base of a valve cusp and holes indicating a portion to be a suture allowance, and a wing forming part, which is outside the valve cusp base forming part and corresponds to a hole or holes for forming a wing part to be fixed to a blood vessel. In addition, this template has a plurality of scale marks that indicate the points where suturing is performed during a procedure on the outside of the valve cusp base forming part. When forming an aortic valve from the pericardium, this template is used to draw lines along the valve cusp base forming part with a surgical skin marker and to draw marks at the hole positions of the wing forming part. Thereafter, the pericardium is cut with reference to the drawn marks at the hole positions of the wing forming part and the lines, by which a valve cusp material to be a valve cusp is formed.

### CITATION LIST

### Patent Document

### Patent Document 1: Japanese Patent Application Laid-Open No. 2015-181521

### SUMMARY OF THE INVENTION

### Problems to be Solved by the Invention

In the case of the template disclosed in Patent Document 1, marks are drawn with reference to the scale marks provided on the outside of the valve cusp base forming part, by which the drawn marks serve as marks for positions where the valve cusp material is sutured to the aorta. Since the position of the edge of the valve cusp base forming part corresponds to a part to be a suture allowance, however, drawing the marks at the actual suturing positions requires determining the suture allowance from the scale marks present along the edge of the valve cusp base forming part by eye before drawing the marks at positions separated in the radial direction by the suture allowance determined by eye. In this case, due to the lack of skill of a user or the misunderstanding of the meaning of the scale marks, it was likely that the marks would not be drawn at appropriate positions. Suturing according to inappropriately positioned marks may affect the function of the valve cusp formed by the suture.

An object of the present invention is to assist in drawing marks indicating suture positions at appropriate positions in a pericardium.

### Means for Solving the Problems

The present invention provides a template including: a plurality of first holes for drawing lines indicating positions at which a membrane is to be cut; and a plurality of second holes that are provided at a predetermined distance from the plurality of first holes and are for use in drawing marks at suture positions where the cut membrane is to be sutured.

The present invention may be configured so that a hole for drawing a mark at a first position that is to be a suturing start position on the membrane cut along the line among the plurality of second holes differs in at least shape or size from a hole for drawing a mark at a position other than the first position.

The present invention may be configured so that one of the plurality of second holes is a hole for drawing a mark at a second position that is to be used as a position at which the membrane is sutured to an adjacent valve cusp when suturing the membrane cut along the line.

The present invention may be configured so that the hole for drawing the mark at the second position differs in at least shape or size from a hole for drawing a mark at a position other than the second position.

The present invention may be configured so that the template has the second hole between the second hole for drawing the mark at the second position and the first hole.

The present invention may be configured so that the portion other than the first holes and the second holes is transparent.

### Advantageous Effect of the Invention

The present invention enables assisting in drawing marks indicating suture positions at appropriate positions in a pericardium.

### BRIEF DESCRIPTION OF THE DRAWINGS

FIG. 1 is a diagram illustrating an example of a template 1 according to the present invention.

FIG. 2 is an enlarged view of a forming part 2.

FIG. 3 is a diagram illustrating an example of lines and marks drawn on the pericardium by using the template 1 and a skin marker 5.

FIG. 4 is a diagram illustrating an example of a valve cusp material 6 obtained by cutting the pericardium

FIG. 5 is a diagram for describing suturing of the valve cusp material 6 to an aorta 7.

FIG. 6 is a diagram for describing suturinq of the valve cusp material 6 to the aorta 7.

FIG. 7 is a diagram illustrating an example of the forming part 2 according to a modification.

FIG. 8 is a diagram illustrating a template 1A according to the modification.

### MODE FOR CARRYING OUT THE INVENTION

### Embodiments

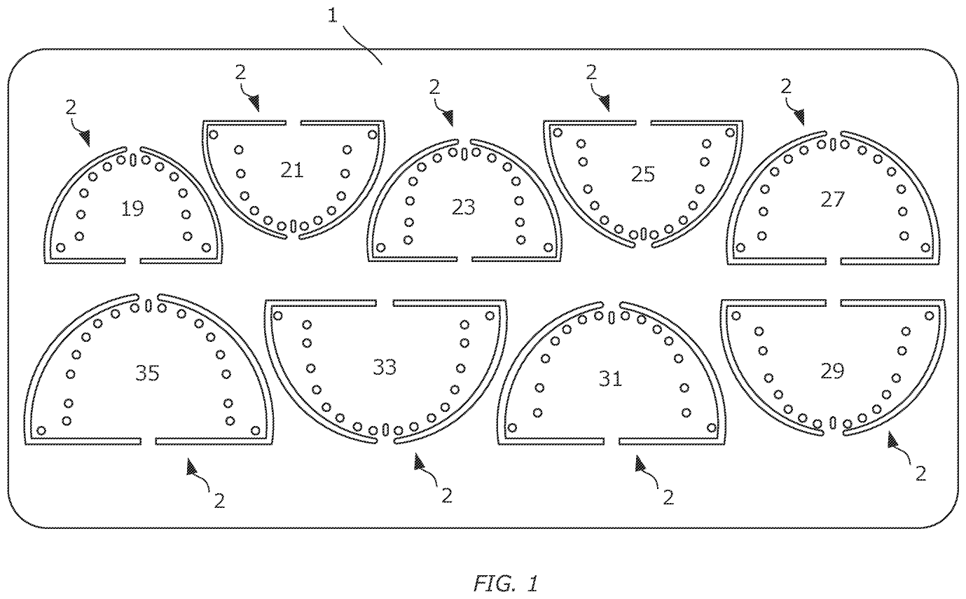

FIG. 1 is a diagram illustrating an example of a template 1 according to the present invention. The template 1 is for use in aortic valvuloplasty and is used to cut the pericardium taken from a patient to form a valve cusp of an aorta of the patient. The template 1 is transparent, so that the pericardium under the template 1 can be seen when placed on the top of the pericardium taken from the patient. The template 1 includes a plurality of forming parts 2 to accommodate various sizes of valve cusps to be formed from the pericardium.

When forming a valve cusp material that is to be used as a valve cusp from the taken pericardium, for example, the size of the valve cusp is measured using a plurality of valve cusp sizers disclosed in Japanese Patent Application Laid-Open No. 2016-54127. The valve cusp sizer is used to measure a distance from one commissure part where the excised valve cusp has been present to the other commissure part on the inner circumference of the blood vessel in the aorta from which the valve cusp was excised. The valve cusp sizer has an arcuate surface when the cylinder is cut at an angle (for example, 120 degrees) corresponding to the central angle of the commissure part of the aortic valve. Regarding the size of the valve cusp sizer, for example, the diameter of the cylindrical shape before being divided into three equal parts is used as a nominal diameter for specifying the size of the valve cusp, and the nominal diameter is an odd number from 17 to 35. The number printed on each forming part 2 indicates the size of the valve cusp and corresponds to the nominal diameter value of the valve cusp sizer described above. The size of the forming part 2 is not limited to the above-described size, and the template 1 may have a configuration including the forming part 2 for a size other than the above-described size. Moreover, the nominal diameter of the valve cusp sizer and the size of the forming part 2 may be even numbers, and may have both odd and even numbers.

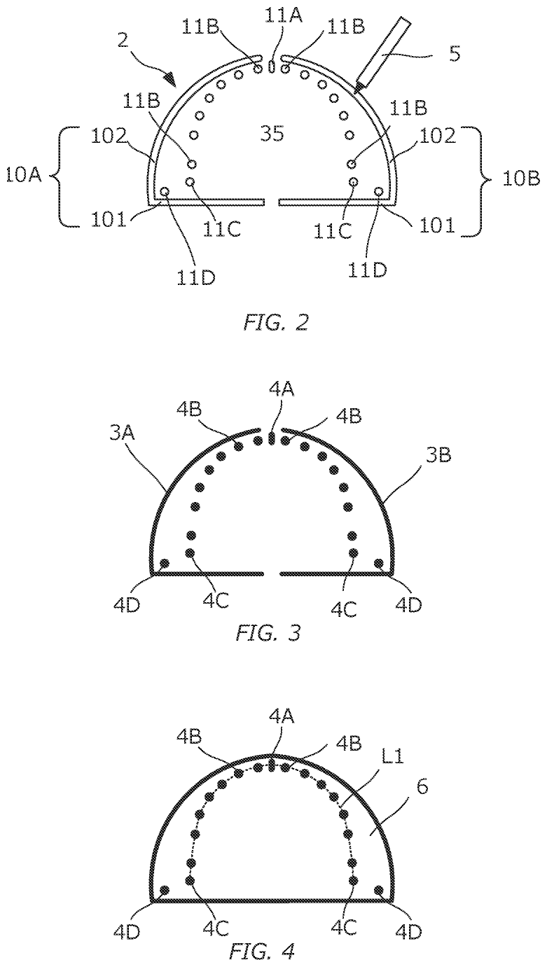

Subsequently, the configuration of each forming part 2 will be described by giving an example of an enlarged forming part 2 illustrated in FIG. 2. Each of the plurality of forming parts 2 has a hole 10A and a hole 10B for drawing lines which indicate the positions at which the pericardium taken from a patient is to be cut. The hole 10A and the hole 10B are merely examples of first holes according to the present invention. In this embodiment, the hole 10A and the hole 10B have a symmetrical relationship. The hole 10A and the hole 10B each have a linear straight line part 101 and an arc part 102 on an arc. The lengths of the straight line part 101 and the arc part 102 are different for each forming part 2.

In addition, each of the plurality of forming parts 2 has a hole 11A, a plurality of holes 11B, holes 11C, and holes 11D for use in drawing marks indicating positions at which valve cusp material cut and formed from the pericardium is sutured to a blood vessel. The hole 11A, the plurality of holes 11B, holes 11C, and holes 11D are examples of the second holes according to the present invention. The hole 11A, the plurality of holes 11B, holes 11C, and holes 11D are provided at a predetermined distance from the hole 10A and the holes 10B.

The hole 11A is a hole for drawing a mark indicating the start position (first position) of suturing the valve cusp material formed from the pericardium, and has a different shape from the hole 11B, the hole 11C, and the hole 11D. Although the hole 11A has an oval shape in this embodiment, the shape is not limited to the oval shape, and any other shape may be applied as long as it is different from the shape of the hole 11B, the hole 11C, and the hole 11D. In addition, the hole 11A may be similar in shape to the hole 11B, the hole 11C, and the hole 11D, and may be larger than the hole 11B, the hole 11C, and the hole 11D.

The number of the plurality of holes 11B between the hole 11A and the hole 11C corresponds to the size of the valve cusp printed on the forming part 2. The positions of the holes 11B correspond to the suture positions in suturing the valve cusp material from the start position of suturing the valve cusp material toward the commissure part.

The hole 11C is for use in drawing a mark indicating the suture position in suturing the valve cusp material to the adjacent valve cusp. Moreover, the hole 11D is for use in drawing a mark indicating the suture position in forming a commissure part.

Subsequently, an example of using the template 1 will be described by giving an example of aortic reconstruction, in which an aortic valve is formed by using a valve cusp material formed from a pericardium taken from a patient's heart. In the aortic reconstruction, the sternum is exposed and then a median sternotomy is performed to take the pericardium from the patient's heart. With the heart exposed, an extracorporeal circulation is performed by a heart-lung machine, the heartbeat is stopped, and thereafter the aortic valve is exposed. On the other hand, the pericardium taken from the heart is expanded by a thread and fixedly immersed in a tissue-fixing solution (for example, a solution containing glutaraldehyde).

Subsequently, what is to be removed from the aorta in the valve cusp of the patient's aortic valve is excised. The size of the excised valve cusp is measured by applying the valve cusp sizer described above to the site where the valve cusp has been excised. In the case where a plurality of valve cusps was excised, the valve cusp sizers applied to each excised site to measure the size of each excised valve cusp. After the completion of measuring the size of the valve cusp, the pericardium is removed from the tissue-fixing solution. Then, the part of the forming part 2 with the length of the arcuate surface of the valve cusp sizer is placed on the fixed pericardium, where the valve cusp sizer matches the distance between the commissure parts of the excised valve cusp.

Then, as illustrated in FIG. 2, the pen tip of a skin marker 5 is inserted into the hole 10A and the hole LOB of the forming part 2, and the skin marker 5 is moved along the hole 10 and the hole 10E to draw lines on the fixed pericardium. This enables drawing of the part of the pericardium that is cut to form the valve cusp material. The lines drawn along the hole 10A and the hole 10B may be continuous lines, dotted lines, or broken lines as the lines only need to be a guide for cutting.

Furthermore, the pen tip of the skin marker 5 is inserted into the hole 11A, the holes 11B, the holes 11C, and the holes 11D to draw marks with the skin marker 5 at the respective hole positions. This enables drawing of marks indicating the suture positions in forming a valve cusp by suturing the valve cusp material obtained by cutting the pericardium to the aorta. Drawing cannot be performed at positions other than the positions of the hole 11A, the holes 11B, the holes 11C, and the holes 11D, that is, at positions where holes are not open in the template 1, and therefore no matter who draws marks, the marks are drawn at appropriate positions since drawing is performed at predetermined positions when viewed from the lines drawn along the hole 10A and the hole 10B.

FIG. 3 is a diagram illustrating an example of lines and marks drawn on the pericardium by using the template 1 and the skin marker 5. A line 3A is a line drawn by using the hole 10A, and a line 3B is a line drawn by using the hole 10B. A mark 4A is a mark drawn by using the hole 11A, and a mark 4B is a mark drawn by using the hole 11B. A mark 4C is a mark drawn by using the hole 11C, and a mark 40 is a mark drawn by using the hole 11D.

Subsequently, the pericardium is cur along the line 3A and the line 3B to obtain a valve cusp material 6 that is to be formed as a valve cusp. FIG. 4 illustrates an example of the valve cusp material 6 obtained by cutting the pericardium. Since the hole 10A and the hole 10B are not connected to each other, lines that serve as a guide for cutting are not drawn between the end portion of the straight line part 101 of the hole 10A and the end portion of the straight line part 101 of the hole 10B, and between the end portion of the arc part 102 of the hole 10A and the end portion of the arc part 102 of the hole 10B, but these parts are cut so as to connect between them. Until being sutured to the aorta, the valve cusp material 6 obtained from the pericardium is soaked in, for example, physiological saline.

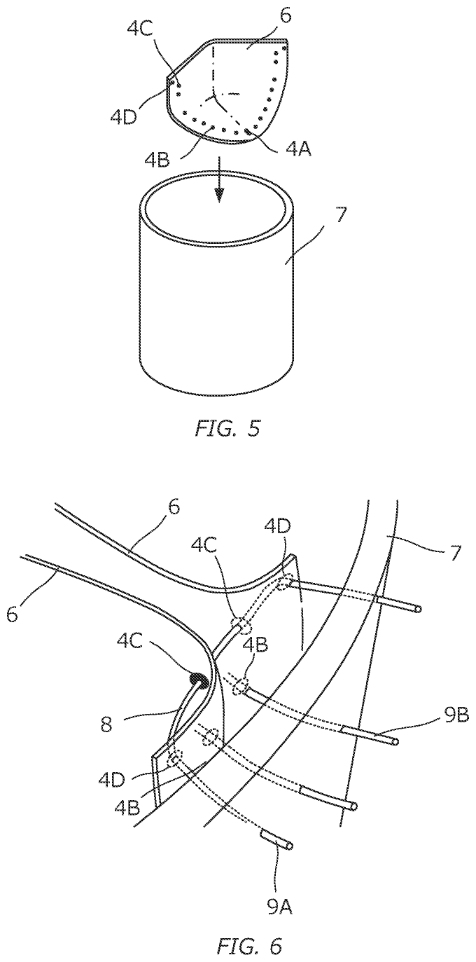

When suturing the valve cusp material 6 to the aorta, the valve cusp material. 6 is inserted into the aorta 7 as illustrated in FIG. 5. In the case of assuming a virtual line L1 connecting the mark 4A to the mark 4C illustrated in FIG. 4 in the valve cusp material 6, the outside of the virtual line L1 serves as a suture allowance when suturing the valve cusp material 6 to the aorta 7. A surgeon threads the mark 4A and sutures the valve cusp material 6 to the aorta 7 with the valve cusp material 6 valley-folded along the virtual line L1 when viewed from above, and sequentially sutures the part along the positions of the marks 4B from the mark 4A toward the commissure part. After continuing the suturing to a portion close to the commissure part, the surgeon sticks the needle out of the aorta 7 from a mark 4B that is closest to the mark 4C.

As for the positions of the mark 4C and the mark 4D, the surgeon sticks a needle (not illustrated) on one end side of a thread 8 from the position of the mark 4C to the position of the mark 4C of the adjacent valve cusp material 6, where the thread 8 is different from the thread used for suturing from the suturing start position to the commissure part and has needles at both ends, as illustrated in FIG. 6. The needle on one end side that penetrates the adjacent valve cusp material 6 protrudes out of the aorta 7 from the position of the mark 4D on the adjacent valve cusp material 6, and the needle (not illustrated) on the other end side of the thread B protrudes out of the aorta 7 from the position of the mark 4D on the valve cusp material 6.

The surgeon applies a pledget to the outside of the aorta 7 and ties a thread 9A, which has been started from the suturing start position and passed through the mark 4B so as to be out of the aorta 7, to a thread 9B, which passed through the mark 4B of the adjacent valve cusp material 6 so as to be out of the aorta 7, on the pledget and then ties the thread 8, which passed through the marks 4D with both ends out of the blood vessel, on the pledger to fix the valve cusp material 6 to the aorta 7.

According to the template 1 of this embodiment, the hole 11A, the holes 11B, the holes 11C, and the holes 11D cause the mark 4A, the marks 4B, the marks 4C, and the marks 4D to be provided at predetermined ideal positions with the line 3A and the line 3B as a reference. A user of the template 1 only needs to position the pen tip of the skin marker 5 in each hole, and the template 1 is configured so that a mark cannot be drawn at a position other than the position corresponding to each of the above holes in the cut pericardium, by which a mark is prevented from being drawn at an inappropriate position.

### Modifications

Although the embodiments of the present invention have been described above, the present invention is not limited to the above-described embodiments and may be carried out in various other modes. For example, the present invention may be carried out by modifying the above embodiments as described below. The embodiments described above and the modifications described below may be respectively combined.

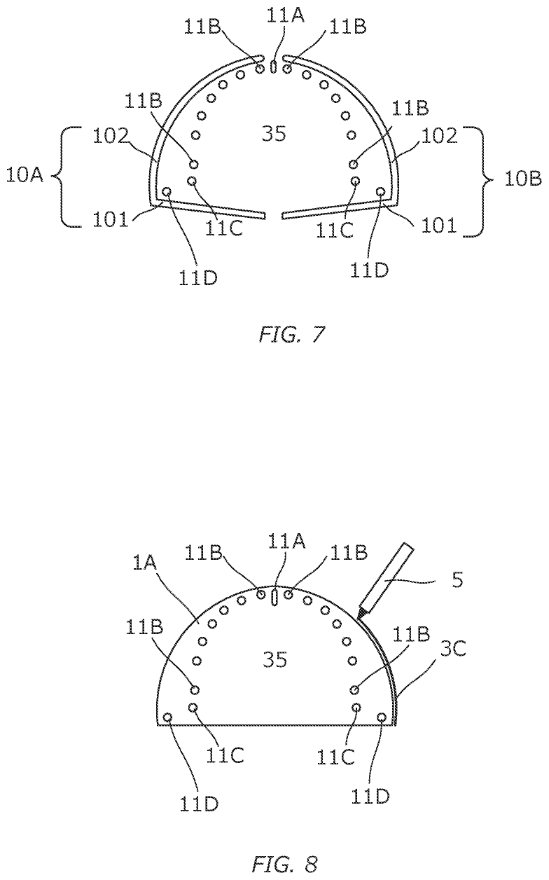

The shapes of the hole 10A and the hole 10B are not limited to the shapes illustrated in FIG. 2, and may be, for example, the shapes illustrated in FIG. 7. The shape of the hole 11C is not limited to the shape illustrated in FIG. 2, and may be different from the shape of the hole 11B, for example. In addition, the shape of the hole 11D may be different from the shape of the hole 10B and the hole 10C. Further, the hole 11C may be different in size from the hole 11B and the hole 11D, and the hole 11D may also be different in size from the hole 11 and the hole 11C.

The template for forming the valve cusp material 6 and for drawing the marks indicating the suture positions on the valve cusp material 6 is not limited to the configuration of the above-described embodiments. FIG. 8 is a diagram illustrating a template 1A according to a modification of the present invention. The template 1A is transparent, and a plurality of templates 1A are provided to accommodate various sizes of valve cusps to be formed. Note that the template 1A does not always need to be transparent. The number printed on each of the plurality of templates 1A is a number indicating the size of the valve cusp, and corresponds to the length of the arcuate surface of the valve cusp sizer described above. The shape of the template 1A is similar to the shape of the valve cusp material 6 illustrated in FIG. 4, and the template 1A has a hole 11A, holes 113, holes 11C, and holes 11D for use in drawing marks indicating the positions at which the cut and formed valve cusp material 6 is sutured to a blood vessel.

In the case of using the template 1A, a line 3C is drawn on the pericardium by moving the skin marker 5 along the edge of the template 1A. This enables drawing of a part of the pericardium that is to be cut to form the valve cusp material 6. Moreover, the pen rip of the skin marker 5 is inserted into the hole 11A, the holes 11B, the holes 11C, and the holes 11D to draw the mark 4A, the marks 48, the marks 4C, and the marks 4D at the respective hole positions with the skin marker 5. This enables drawing of marks indicating the suture positions when the valve cusp material 6 is sutured to the aorta 7 to form a valve cusp. According to the template 1A, the template 1A is not brought into contact with parts other than the cut parts, thereby enabling lines to be easily drawn with the skin marker 5.

### DESCRIPTION OF REFERENCE NUMERALS

1, 1A: templates
2: forming part
3A, 3B: lines
4A, 4B, 4C, 4D: marks
5: skin marker
6: valve cusp material
7: aorta
8: thread
9A, 9B: threads
10A, 10B: holes (first holes)
11A, 11B, 11C, 11D: holes (second holes)
101: straight line part
102: arc part
L1: virtual line

1. A template comprising:
a plurality of first holes for drawing lines indicating positions at which a membrane is to be cut; and
a plurality of second holes that are provided at a predetermined distance from the plurality of first holes and are for use in drawing marks at suture positions where the cut membrane is to be sutured.

2. The template according to claim 1, wherein a hole for drawing a mark at a first position that is to be a suturing start position on the membrane cut along the line among the plurality of second holes differs in at least shape or size from a hole for drawing a mark at a position other than the first position.

3. The template according to claim 1, wherein one of the plurality of second holes is a hole for drawing a mark at a second position that is to be used as a position at which the membrane is sutured to an adjacent valve cusp when suturing the membrane cut along the line.

4. The template according to claim 3, wherein the hole for drawing the mark at the second position differs in at least shape or size from a hole for drawing a mark at a position other than the second position.

5. The template according to claim 4, wherein the template has the second hole between the second hole for drawing the mark at the second position and the first hole.

6. The template according to claim 1, wherein the portion other than the first holes and the second holes is transparent.
